(19) United States
(12) Patent Application Publication
(10) Pub. No.: US 2021/0007630 A1
(43) Pub. Date: Jan. 14, 2021

(54) Prone Play Activity Center

(71) Applicant: Virginia Commonwealth University, Richmond, VA (US)

(72) Inventors: Peter Pidcoe, Mechanicsville, VA (US); Stacey Dusing, Richmond, VA (US)

(21) Appl. No.: 16/508,533

(22) Filed: Jul. 11, 2019

(51) Int. Cl. A61B 5/11; A63H 33/00; A63H 5/00

(52) U.S. Cl. CPC A61B 5/1116; A61B 5/1121; A63H 2200/00; A63H 5/00; A61B 2503/04; A63H 33/006

(57) ABSTRACT

An exemplary embodiment includes an instrumented play center. The play center is a rehabilitation device that 1) positively reinforces head lifting during prone (tummy) play in typically developing infants and children with developmental delays; 2) increases opportunities for prone play; and 3) provides early intervention, physical and occupational therapists a rehabilitation device that can be customized to provide the “just right” challenge promoting prone motor control through interactive play. The play center provides an child with positive reinforcement by activating a toy when the child in prone lifts his/her head over an adjustable threshold.

### FIELD OF THE INVENTION

The invention generally relates to devices and methods for improving infant health and activity in prone.

### BACKGROUND

Prone play is important for infants to develop strength and coordination in the neck, trunk and upper extremities muscles. It provides natural opportunities for the acquisition of head control, reaching, sitting and prone mobility. Prone mobility is one of the early forms of locomotion in infants, expanding opportunities for them to explore and learn. However, the dynamics of prone mobility are complex and require constant change and adaptations in infants' prone motor control.

The American Academy of Pediatrics (AAP) recommends that “parents and caregivers should incorporate supervised, awake ‘prone play’ in their infant's daily routine to support motor development and minimize the risk of positional head deformities.” However, approximately 70% of 4-5 months old infants are spending more time in supine, supported sitting or being held (x=8.9 hours, SD=1.26) compared to prone position (x=1.2 hours, SD=1.1) during the day. It is estimated that 20% of 4 million infants born in 2013 would experience some degree of positional skull deformation. Studies have suggested excessive supine lying (β=2.8; 95% CI: 2.23-3.32) and limited prone play time (β=0.9; 95% CI: 1.53-0.22) in infants' routine as factors associated with the risk of positional plagiocephaly. Yet only 26% of 6 month olds practice prone play daily.

Infants' poor tolerance for prone position, biomechanical challenges imposed by prone position on the musculoskeletal system and parents' hesitation towards prone play creates a cycle leading to reduced practice of motor skills in prone. Around 53% of parents have reported their infant being intolerant, or crying excessively, in prone position resulting in parents further limiting their prone play. Biomechanically, an infant's body is top-heavy due to the proportionally large size of the head. This displaces their center of gravity forward and centered on the sternum in prone. Infants must use their still developing weak neck and trunk muscles to shift their weight posteriorly to lift their upper body in prone to look around and explore. According to the ecological theory of motor learning, practice along with knowledge of performance (feedback received during the movement) and knowledge of results (feedback received after the movement) is beneficial for motor learning. For infants to develop prone motor skills it is necessary to learn the ability to shift their center of gravity posteriorly and gain strength through practice.

Due to infants' intolerance for prone position, parents, including those parents familiar with AAP's “back to sleep and prone to play” recommendations, find it challenging to incorporate prone play in their infant's daily routine. As a short term solution parents may decide to move infants out of prone play to a position that is not challenging such as supine or put them on a swing. In the long term, parents may perceive practicing prone play with their infant as a burden and completely discontinue the practice of prone play in their infants' routine. This cycle of limited practice, persistent weakness, and poor tolerance contributes to findings of infants spending ˜75% of the wakeful period of the day either being held or in an equipment such as a seating device.

Current approaches to increase prone motor skills and improve tolerance for prone play include educating parents through brochures and using commercially available prone positional supports such as U shaped pillows and play gyms. Lack of scientific rigor in the prone play recommendations leads to poor implementation of prone play during infancy as health care providers and parents are not clear about the amount of time suggested by the guidelines or how best to implement the prone play recommendations.

U-shaped pillows or towel rolls under the chest are positional supports commonly used by parents to increase prone play time. However, positional supports do not positively reinforce the infant's efforts to lift the head higher or lift the head for a longer period of time.

Prone positional supports, like any other equipment (seating device, walkers) restricts an infant's motor workspace, or the area in which an infant practices a variety of motor skills and learns to make a selection of the most efficient one. Equipment that does not provide any reinforcement makes the infant passive and results in poor performance in measures assessing motor development.

Commercial play gyms often combine a prone pillow with toys or mirrors located in the infants' field of vision when placed in prone. While this type of commercial play gym may have toys, the toy's activity is not related to infant's movement Likewise, the toys are often accessible with the head down in prone or in supine or sitting as well. Without positive reinforcement during prone play, infants may disengage, fuss or roll into supine.

Current education programs and “tummy time toys” do not address the need for an easily implemented prone play strategy that can be implemented by parents or daycares. A need exists for an evidence based intervention approach usable by physical therapists, early intervention providers, parents and daycares to increase tolerance for prone play and improve prone motor skills.

Motor behaviors that have been studied most commonly with the use of associative learning paradigms in infants are kicking and reaching. For example, Sargent B, Reimann H, Kubo M, Fetters L. Quantifying Learning in Young Infants: Tracking Leg Actions During a Discovery-learning Task. J Vis Exp. 2015;(100):e52841 uses wearable sensors to quantify infants' kicking response and uses computational technology to create an effect on a mobile when the infant kicks to a certain height. Reaching and kicking behaviors share some properties such as infants early in development move their upper and lower limbs spontaneously in a rhythmic manner.

In Williams J L, Corbetta D. Assessing the impact of movement consequences on the development of early reaching in infancy. Front Psychol. 2016; 7(APR):1-15, toys that moved and sounded only upon contacts encouraged 3 month old infants to contact more and practice reaching and object exploration.

Despite research related to kicking and reaching, there has been limited research on mechanics of infant's prone motor behaviors and learning. Some common factors identified in the literature that are responsible for the decline of technology in pediatric rehabilitation are the technology being bulky, not aesthetically pleasing, parents negatively feeling that their child is “wired up,” and the child growing out of device.

### SUMMARY

An exemplary embodiment includes an instrumented infant play center. The play center is a rehabilitation device that 1) positively reinforces head lifting during prone (tummy) play in typically developing infants and infants with developmental delays; 2) increases opportunities for prone play; and 3) provides early intervention, physical and occupational therapists a rehabilitation device that can be customized to provide the “just right” challenge promoting prone motor control through interactive play. The play center provides an infant with positive reinforcement by activating a toy when the infant positioned in prone lifts his/her head over an adjustable threshold. The adjustable threshold allows parents or therapists to challenge the infant to work to his/her highest capacity, consistent with the current evidence on rehabilitation techniques to advance motor control. Parents or therapists can use the device to calculate the average height of head lifts and then set the intervention threshold. The adult user may also use these measures to quantify changes in infant's prone activity over time.

An exemplary instrumented infant play center may comprise one or multiple modes of operation. For example, an embodiment may comprise two modes: continuous mode and interval mode. In the continuous mode the toy activates when the infant's head is at or above the threshold and will turn off when the infant's head is below the threshold. In the interval mode, the toy activates when the infant's head is at or above the threshold, but the toy will turn off after a certain period of time or interval length even if the infant's head remains above the threshold. A controller for the play center may be configured to operate or be operable in an interval mode by which after activation of the toy, the positive reinforcement feedback device is deactivated after a predetermined time interval if during the time interval the head position has not fallen below the activation threshold and subsequently exceeded the activation threshold.

An exemplary play center comprises ultrasonic sensors, a microcontroller with associated electronics, and a dancing/singing toy. Other elements may include power provision (e.g., battery system), a display unit/box, and a play mat. The ultrasonic sensors locate the elevation of infants' head in space and sends the heads' distance from the sensor to the microcontroller. The microcontroller uses this information to match with stored settings and activates the toy if conditions are met. In its simplest mode, the device functions with its intended purpose to activate a toy when an infant's head is lifted to a certain height, reinforcing specific motor activity, while recording these events. More complicated control algorithms include selective deactivation of the toy and temporal controls to promote/encourage selected infant behaviors.

According to an aspects of some embodiments, an interactive device is provided that may be used in the home, therapy clinics and even daycares to increase the dose of positively reinforced prone play in an infant's daily routine.

An exemplary device provides positive reinforcement in response to an infant's movements in prone, engages them with a toy that responses to their movement and can be adjusted to increase the challenge as the infant skills increase. Utilization of such a device is likely to increase prone tolerance and duration of prone play while reducing the risk of plagiocephaly and delayed development for infants.

According to an aspect of some embodiments, principles of associative learning are combined with technology to produce a learning paradigm instrument that may be used in the future to enhance prone motor control of infants. To the inventors' knowledge, associative learning has never been tested in prone position in infants.

According to an aspect of some embodiments, an innovative integration is provided of features of infant development and motor learning with technology to advance developmental science and pediatric rehabilitation. Motor learning is described in the literature as a “set of processes associated with practice or experience leading to a relatively permanent change in the capability for producing skilled action”. Based on the contemporary view of infant development, infants learn to acquire a skill through their interaction with the environment. Exploration through self-initiated movement provides natural opportunities to infants to find a purpose in their actions and gain the knowledge or perception of the movement itself and the environment.

Some embodiments are devices or comprise devices. Some embodiments are or comprise assessment, intervention, and/or training protocols.

Rehabilitation devices according to some embodiments may be configured to assess infant movement and function and optimize the device response to promote particular movement, functions, and/or activities of the infant. Used in physical therapy (PT) contexts, some embodiments may be used to enhance the effectiveness of PT assessments and interventions for child development and learning.

Some embodiments include an ability to measure infant learning and to provide infants with opportunities to practice a behavior utilizing motor and cognitive skill, with reinforcement, which might improve tolerance for prone play and advance their prone motor skills.

Caretaker compliance with device use instructions may have a significant impact on the effectiveness of an exemplary play center. Parent and child friendly features and aspects, as well as exemplary use procedures, are included to promote compliance.

Two examples are provided at the end of the Detailed Description below. The first example determines whether 3-6 months old infants can demonstrate associative learning in prone. The second example evaluates the feasibility of using a high technology intervention based on the principles of motor learning and associative learning to increase infants' prone tolerance and improve motor outcomes.

Findings from the examples suggest the following: 1) infants can independently discover the association between their head and upper body movements and the activation of a toy, 2) during the discovery/associative learning process infants learned to change their motor behavior in prone to keep the toy on for longer periods of time, 3) both a high technology intervention that uses an automated play center to positively reinforce the infants head and upper body movements and a dose matched low tech educational condition based on usual care are feasible interventions, and 4) measurement of changes in the infant's prone tolerance and motor development following the high tech and educational intervention is feasible.

The broader advantages of some embodiments are to 1) leverage the learning mechanisms underpinning the development of prone motor control in both typically developing infants and infants with developmental delays and 2) develop interventions to improve infant tolerance for prone play and positively impact motor development.

Example 1's purpose was to understand if 3-6 months old infants can demonstrate 1) short term learning of an association between their upper body, head and torso movements and activation of a toy while in prone position and 2) retention of the association learned on day 1, 24 hours later. Twenty eight, 3-6 month old, typically developing infants were tested for 2 consecutive days on their ability to learn the causal relationship between lifting their head in prone and activating a toy. An instrumented play gym was used for 2 consecutive days using the same protocol on each day: 1) Baseline phase (2 minutes), toy won't activate in response to infant movement 2) Acquisition phase (8 minutes), toy activates for maximum of 10 seconds if the infant's head is above a threshold. Infants were categorized as 1) short term learners if the frequency of toy reactivations (FTR) or total duration toy was on (DTO) during Acquisition was 1.5 times Baseline and 2) retainers of the association learned on day 1, if FTR or DTO on day 2 was 1.5 times day 1's Baseline. Of the 28 infants, data of 22 infants was included for analysis. Fourteen infants were categorized as short term learners on day 1. Of the short term learners on day 1, 3 infants demonstrated retention of the association. Example 1 supports a finding that when 3-6 month old infants born at full term are presented with a task of raising their head to a certain height to activate a toy, a majority of infants tend to learn the association of their movement with the activation of the toy.

Example 2's purpose was to determine the feasibility of completing a clinical trial comparing usual care to a high-tech intervention to increase tolerance for prone and improve prone motor skills. The proposed high tech intervention included 1) positive reinforcement provided when infants' activate a toy by raising their head above a threshold and 2.) Just Right Challenge (JRC) by challenging infants to a slight higher threshold each time they achieve a target. Ten full-term infants with poor prone tolerance were randomized to the high-tech or the low-tech, education group. Parents and infants in each group participated in a 3 week intervention with 4 PT visits and 15 parent sessions. Data on intervention frequency and parent feedback were used to determine the feasibility of the high-tech intervention. Effect sizes were calculated for motor and prone tolerance measure at baseline and end of intervention. Infants received an average of 93% of the anticipated high-tech intervention. Parents had high adherence to one of the 2 key components of the intervention and independently used the high technology for average 18 minutes per day. Effect sizes were large for the motor development and prone tolerance measures and in the anticipated direction. The example supports finding that the high-tech intervention is a feasible intervention.

### BRIEF DESCRIPTION OF THE DRAWINGS

FIG. 1A is a side view of an exemplary instrumented infant play center with an infant.

FIG. 1B is a top plan view of the exemplary play center.

FIG. 2A is a flowchart of an exemplary method.

FIG. 2B is a diagram depicting the mechanism of change in infant behavior and training according to some exemplary embodiments.

FIG. 3 is a graph of within group comparison of short term learners FTRs at 5 phases of testing (Baseline, Acq 1, Acq 2, Acq 3 and Acq 4 (n=12). Error bar represents standard deviation.

FIG. 4 is a graph of within group comparison of the short term learners DTO at 5 phases of testing (Baseline, Acq 1, Acq 2, Acq 3 and Acq 4 (n=12)). Error bar represents standard deviation.

FIG. 5A shows frequency of toy reactivations for short-term learners.

FIG. 5B shows frequency of toy reactivations for nonlearners.

FIG. 5C shows frequency of toy reactivations for retainers.

FIG. 5D shows frequency of toy reactivations for nonretainers.

FIG. 6A is duration of head above threshold and toy activation for short-term learners.

FIG. 6B is duration of head above threshold and toy activation for nonlearners.

FIG. 6C is duration of head above threshold and toy activation for retainers.

FIG. 6D is duration of head above threshold and toy activation for nonretainers.

FIG. 7 is a group comparison of progression in prone play over 3 weeks of intervention.

FIG. 8 is mean change in prone tolerance from baseline to EOI. Error bars represents standard deviation.

FIG. 9 is mean change in GMFM-66 from baseline to End of intervention. Error bars represents standard deviation.

FIG. 10 is mean change in AIMS scores from baseline to End of Intervention. Error bars represents standard deviation.

### DETAILED DESCRIPTION

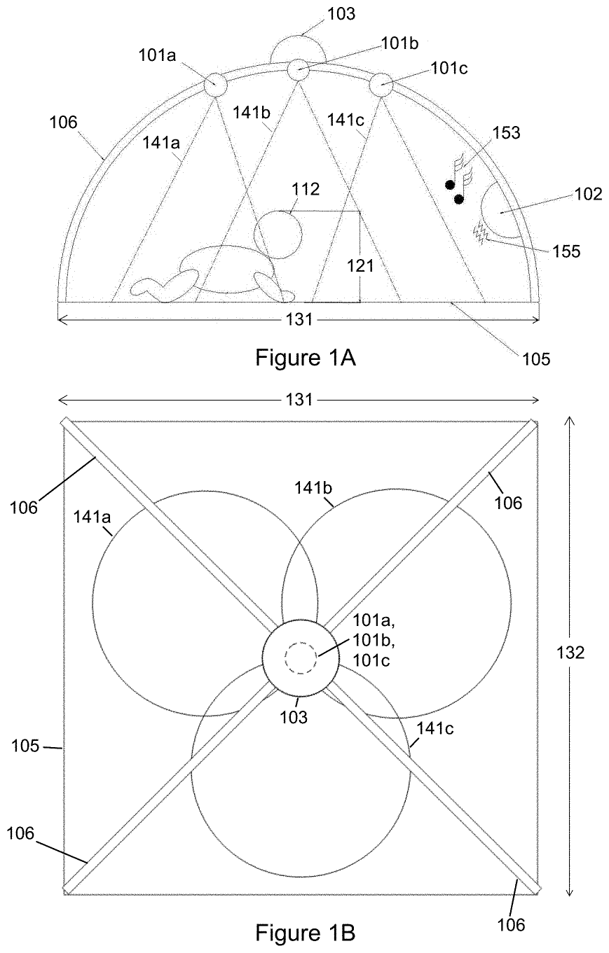

FIGS. 1A and 1B show an exemplary instrumented infant play center 100 depicting an infant in use thereof (for clarity the infant is omitted from FIG. 1B). For purposes of this disclosure, the terms “activity center,” “play center,” and “play gym” may be used interchangeably. The term “infant” is generally used to refer to persons 0 to 1 year old. Play centers and methods according to the invention may be used for persons other than infants (e.g., babies and children older than 1 year old), although typically developing children are expected to move beyond prone by ages in excess of 1 year. Children with disabilities may still benefit from embodiments disclosed herein despite reaching ages in excess of 1 year.

The exemplary play center 100 is based on the principles of associative learning. Associative learning (AL) may be described as the ability to discover a causal relationship between two or more events. According to operant conditioning (OC), which is a form of AL, a behavior can be encouraged or suppressed by associating it with a positive or negative consequence (rewards/punishments). Operant conditioning techniques have never before been used with head lifting in prone. Exemplary embodiments employ the principles of operant conditioning according to which a behavior can be encouraged by associating it with a positive experience or reinforcement. An exemplary device may be configured to use operant conditioning to modify motor behaviors, teaching infants to bring about a change in their prone motor behavior. An exemplary play center 100 provides an artificial environment where an infant's prone motor behavior is precisely associated with an effect. In particular, some embodiments associate infants' upper body movements in prone with a positive reinforcement.

The exemplary play center 100 comprises one or more sensors 101 (101a, 101b, 101c), a positive reinforcement feedback device 102, and a controller 103. The sensors may be arranged at different positions on the play center. To show two non-limiting alternatives, FIG. 1A shows sensors 101a, 101b, and 101c at different positions on the play center 100 including at the controller 103 and positioned apart from the controller 103 along supports 106. By contrast, FIG. 1B shows all three sensors 101a, 101b, 101c located in the center pod of the controller 103.

The sensors 101 are configured for monitoring the position of the infant's head 112 while the infant is in prone. Prone is generally defined as lying with the body oriented “face-down.” A body orientation of “face-down” does not preclude the possibility of the head and face (that is the front of the head) being lifted with the neck and upper back so that the face faces forward despite the body facing down. The sensors 101 may be, for example, infrared or ultrasonic sensors. Ultrasonic provides a higher resolution than infrared and therefore may be preferred in some embodiments which benefit from a higher resolution. Other sensor types may be used in addition or as an alternative to ultrasonic and infrared. One alternative is low-power laser, though this option may require certain precautions be taken to avoid a risk of damage to the eyes of the infant. At least one sensor 101b may be mounted above an anticipated position of an infant's head 112. The sensor(s) 100 are configured to locate the head position in space. In some embodiments, the sensor(s) may simply be configured to detect the highest entity in the monitored space; for infants who cannot independently move out of prone and who cannot reach above shoulder level while in prone, the head is the highest entity. The sensor(s) 100 precipitate activation of a toy or other positive reinforcement feedback device 102 when an infant raises its head to or above a certain height. The activation threshold may be a particular value set or adjusted by an (adult) user and/or the controller based on a preconfigured algorithm. The activation threshold may be a range of values, e.g., having upper and lower bounds with some non-zero range of heights therebetween. Activation may be triggered whenever the height 121 of the infant head 112 is within the threshold range. Whether a threshold takes the form of a single value or a range of values, the value or range of values may be adjusted or adjustable.

The positive reinforcement feedback device 102 is a device configured to provide some form(s) of feedback to an infant when one or more conditions are met. A positive reinforcement feedback device 102 may be a toy, for example. For convenience of discussion, “toy” may be used in many exemplary illustrations in this disclosure. However some positive reinforcement feedback devices may be something other than a toy. Some positive reinforcement devices may be educational tools or household items appropriately configured for use with or by an infant. Embodiments may include the capability of substituting or swapping out different toys, permitting a degree of customization tailored to an individual child's interests. For example, some exemplary embodiments may offer a choice among a roll-and-glow monkey toy, dance-and-move bug toy, and a mobile. Any of these toys may have features of glowing, singing, and/or moving. For activation purposes by the controller, the toy(s) may have a wired or wireless connection for receiving control signals that cause activation and deactivation.

In general, a positive reinforcement feedback device 102 may be described according to at least two states: activated/on, and deactivated/off. In some embodiments a feedback device may have more than two states or settings so as to provide more graduations or levels of feedback. A feedback device that is a toy may have functionality to sing and/or dance, that is, provide audible feedback 153 and/or visual feedback 155. Other forms of feedback, e.g., tactile, kinesthetic, olfactory, gustatory, thermal, etc. may also or alternatively be used in some embodiments as a modality of feedback supplied to the infant under predetermined conditions.

The controller is configured to activate the positive reinforcement feedback device when the monitored head position is detected as exceeding a threshold. The threshold may be adjusted based on, for example, fitness or ability goals tailored to specific infants, or the pace of progress of a given infant.

A play center 100 may also include a floor, bottom, or mat 105. In a state of use the infant may be confined to the mat 105, e.g., required to remain within the boundaries of the mat 105. The mat 105 may define the boundaries in which the sensor(s) 101 are configured to detect an infant's head 112 so that the head's position and/or head height 121 may be determined.

A play center 100 may or may not include sidewalls, barriers, or other boundary restrictive elements to confine and/or protect the infant. Some play centers may be configured to attach (be attachable to) commercial play pens, play gyms, playards, and like devices which are made to provide a safe space for a child, with or without boundaries.

A play center 100 may further include one or more supports 106. The sensors 101 may be attached or attachable to the supports 106 such that the sensors are maintained at particular positions above the mat 105 (and above the ground, and above the infant during use). Other elements such as the controller 103 and feedback device 102 may also be attached or attachable to the supports 106 but are not necessarily attached to the supports 106.

The supports 106 and mat 105, among other elements of the play center 100, may be deformable or susceptible to disassembly to permit the play center 100 to be compactly stored when not in use.

The configuration of sensors 101 is such that at no anticipated infant head height 121 in prone are the sensors 101 unable to sense the infant head position in space. The number and position of the sensors is selected based on the anticipated play space of an infant. In the figures, the play zone defined by the mat 105 is rectangular (in particular, square) with dimensions 131 and 132. Other mat shapes (e.g., polygonal, circular) may also be used. The detection area of a single sensor (e.g., an infrared or ultrasonic sensor) may be in the shape of a cone or elliptical cone, as is illustrated in the figures by detection areas 141a, 141b, and 141c, corresponding respectively with sensors, 101a, 101b, and 101c. Other detection area configurations are also possible, depending on the specific sensor(s) employed. Depending on the particular embodiment, a single sensor (e.g., with a conical detection area) may or may not be sufficient to detect all anticipated locations of an infant's head. In the illustrated example, a plurality of sensors are used and arranged at different angles with respect to the play space such the cumulative coverage of the respective detection areas includes all anticipated locations of an infant head during use. In the figures, the sensor detection areas 141a, 141b, 141c overlap and give coverage of the anticipated play zone within by the boundaries of the mat 105. The height of the sensors may also be set to be at least 0 inches based on the maximum anticipated height 121 of head position in prone, e.g., 20 inches.

To maximize the likelihood of success in operant conditioning of an infant, exemplary embodiments emphasize accurate tracking of the infant's head position over time and timely activation or deactivation of the feedback device on the basis of the infant's head position or change in head position within a particular duration of time. Meaningful change in infant tolerance for prone and maximizing outcomes may also depend in part on dose (“how much” prone play, or “how early” an age at which regimented prone play is conducted) which may be set according to exemplary methods of use for an exemplary device 100.

The controller 103 may be or include one or more controllers, e.g., a microcontroller, configured to process the signals from the sensor(s) 101 to determine the infant head position and compare that position against a predetermined threshold. Depending on the relationship of detected head position and the threshold, the one or more controllers may transmit a signal to activate or deactivate the dancing/singing toy. In some circumstances the one or more controls may not transmit any new signal or change in signal, leaving the toy in its existing state (which may be activated or deactivated).

The controller(s) may be connected to storage or an external computer or network of computing devices that allow for recording of data over time. Recorded data may include but is not necessarily limited to: 1) Head lift height (HH), distance from the highest point on the infant's head to the floor, 2) Average head lift height (AHH), average HH during a trial (e.g., a duration of repeated uses, altogether spanning several days, several weeks, several months, or several years), 3) Frequency of infant achieving head height at or above the threshold (FAT), 4) Total duration the infant achieves head height at or above the threshold, and 5) total duration the toy was on (DTO). The table below gives a non-exhaustive list of factors or elements which may be recorded, determined, processed, or relied upon during a protocol of use of an exemplary device. A data storage unit may also be used to track the amount of time a play center is used and information on the activity of infants in the play center.

TABLE 1
Terms and abbreviations used in the protocol

| Term (abbreviation) | Definition | How obtained/calculated |
|---|---|---|
| Head lift height (HH) | Distance from the highest | Calculated by the play center |
| | point on the infant's head to | |
| | floor. | |
| Average head lift height | Infant's average head lift | Calculated by the play center |
| (AHH) | height during a trial. | or with data from the play |
| | | center |
| Threshold height (TH) | Height set by an adult user at | Equals AHH during a Pre- |
| | which the toy turns ON. | baseline trial |
| At or Above Threshold (AT) | Infant's head is equal to or | |
| | higher than a threshold height | |
| Frequency of AT (FAT) | Number of times infant | Calculated by the play center |
| | achieves AT | or with data from the play |
| | | center |
| Duration AT (DAT) | Duration of an episode of AT | Calculated by the play center |
| | during a trial | or with data from the play |
| | | center |
| Total DAT | Sum of all DAT during a trial | Calculated by the play center |
| | | or with data from the play |
| | | center |
| Average DAT | Average duration of time | Total DAT/FAT |
| | infant achieves AT during a | |
| | trial | |
| Interval Length(IL) | Maximum duration of toy | Set by an adult user during |
| | activation per AT | the Interval mode |
| Duration of Toy ON (DTO) | Duration of a continuous | DTO = DAT with a |
| | episode of AT during the trial | maximum of IL |
| | with a maximum equivalent | |
| | to the IL. | |
| Frequency of toy | Frequency of reactivations | computing software (e.g., |
| reactivations (FTR) | after meeting the IL | Matlab) may be used to |
| | | calculate the FTR |
| Frequency based learning | | FTR_baseline*1.5 < FTR_AQ |
| criteria | | (any 2 of the final 2 |
| | | acquisition phases) |
| Duration based learning | | DTO_baseline*1.5 < |
| criteria | | DTO_AQ(any 2 of the final |
| | | 3 acquisition phases) |

An exemplary instrumented infant play center may comprise one or multiple modes of operation. For example, an embodiment may comprise two modes: continuous mode, and interval mode. In the continuous mode the toy activates when the infant's head is at or above the threshold and will turn off when the infant's head is below the threshold. In the interval mode, the toy activates when the infant's head is at or above the threshold, but the toy will turn off after a certain period of time or interval length even if the infant's head remains above the threshold. For example, if the interval mode is ON and is set at 10 seconds, the toy will activate when the infant's head is at or above the threshold (AT) and will turn off when the infant is below the threshold or after 10 seconds of time with the toy on, even if the infants head is still at or above the threshold. To reactivate the toy the infant needs to lower his head and then raise his head to the threshold again. While thresholds other than 10 seconds may be used (e.g., in the range of 0-10 seconds; 0, 1, 2, 3, 4, 5, 6, 7, 8, 9, 11, 12, 13, 14, or 15 seconds), 10 seconds is exemplary for balancing the competing considerations of maximizing the amount of positive reinforcement with the limited ability to hold an infant's attention. The threshold may be adjustable and customized to each specific infant based on caretaker observations of the specific infant's average attention span.

Following is an example operation of an exemplary instrumented infant play center. The specific parameters are exemplary but should not be read as necessarily limiting on all embodiments. The sensors locate the position of infants' head in space and records the heads' distance from the floor (HH) every 90 milliseconds (msecs). The microcontroller compares the HH to the controller settings and activates the toy if conditions are met. For example, if the microcontroller is set to activate the toy when the infant's head is ≥10 cm off the floor the toy will turn on when the head is >10 cm and turns off when the infant lowers his head to<10 cm. The microcontroller is connected to a computer that records every 90 msecs.

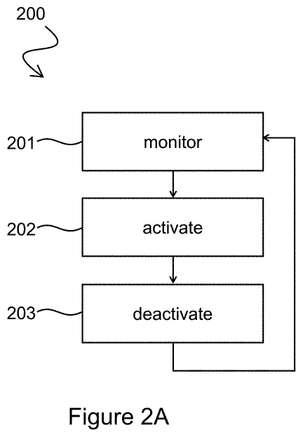

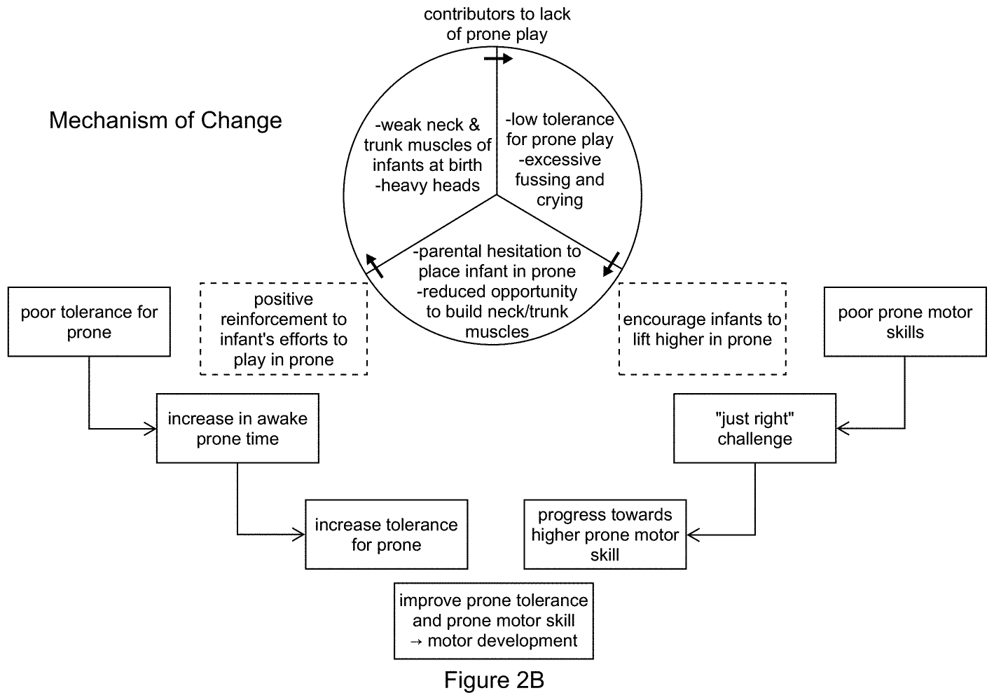

FIG. 2A shows an exemplary method 200. The method 200 comprises, at step 201, monitoring the head position of an infant in prone; at step 202, activating a positive reinforcement feedback device when the head position is detected as exceeding an adjustable threshold; and at step 203, deactivating the feedback device when the condition(s) for activation are no longer met and/or after some predetermined elapsed duration of time.

Following is a non-limiting example of how method 200 may be performed on a more granular level. One or more sensors, e.g. ultrasonic sensors, locate the position of the infant's head in space and records the distance of the head from the floor/mat. A controller compares the recorded head height to a preset parameter or threshold height. The controller activates a toy if the comparison result shows activation condition(s) are met. For example, if the microcontroller is set to activate the toy when the infant's head is ≥10 cm off the floor the toy will turn on when the head is ≥10 cm and turns off when the infant lowers his head to <10 cm.

FIG. 2B is a diagram depicting the mechanism of change in infant behavior and training according to some exemplary embodiments. The dashed boxes represent the “active ingredients” of intervention according to exemplary embodiments. That is, exemplary embodiments provide positive reinforcement to infant's efforts to play in prone and encourage infants to lift higher in prone (e.g., over the course of successive prone play sessions and/or over the case of successive days). Infants which may begin with poor tolerance for prone and/or poor prone motor skills are, through exemplary play centers and intervention methods, encouraged to improve prone tolerance and prone motor skill. This provides infants with overall improved motor development.

Embodiments of the present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### EXAMPLES

### Example 1

### An Innovative Motor Learning Paradigm Combining Technology and Associative Learning to Assess Prone Motor Learning in Infants

The aim of this example is to determine if 3-6 months old infants can demonstrate associative learning by modifying their prone motor control and retain the association 24 hours later. In order to demonstrate associative learning in prone, infants have to modify their existing prone control and adapt to the challenges of the associative learning model proposed in this example.

### Methods

A single group experimental design included 28 typically developing infants born at term who participated at 3-6 months of age. Schraber et al. suggest that for a single group analysis, 10 participants per estimated parameter is acceptable. Schreiber J B, Stage F K, King J, Nora A, Barlow E A. Reporting structural equation modeling and confirmatory factor analysis results: A review. J Educ Res. 2006; 99(6):323-337. doi:10.3200/JOER.99.6.323-338. A minimum sample size of 20 infants was therefore intended on the basis of two parameters: Frequency of Toy Reactivations (FTR) and Duration of Toy On (DTO).

Infants were required to be born at full term (38-42 weeks of gestation) and to be able to lift their head well in prone, but not have the ability to crawl or creep. The prone motor skills criterion was established to ensure that infants' had the motor abilities required to use the play center effectively. In addition, infants were required to have good prone tolerance (not fussing or crying greater than 30 seconds during the 5-minute period in prone while assessing the Alberta Infant Motor Scales—AIMS). This criterion decreases the probability of infants dropping out of the study because they find the testing protocol to be too challenging. Infants were excluded from the study if they were able to move out of prone or pivot, had a brain injury musculoskeletal deformity, genetic syndromes, visual and hearing problems, or condition limiting participation.

### Procedure

All data collection sessions occurred either in the infants' home, a university lab, or the infants daycare center during a time of day the caregiver reported the infant was typically awake and playful.

### Associative Learning Paradigm In Prone

Infants were assessed for associative learning and retention in prone using a play center generally consistent with FIGS. 1A and 1B, with the exception that the toy was not attached to a support but rather was placed adjacent to the mat. Briefly, the experimental play center was a high technology device that included 1) ultrasonic sensors (here, manufactured by HRUSB-MaxSonar) 2) a microcontroller (here, an Arduino Uno), and 3) dancing/singing toy. The sensors located the position of infants' head in space and recorded the heads' distance from the floor. The sensors accuracy and resolution allowed detection of objects of 1 mm to 5 m in size and objects at a distance of 30 cm to 5 m. The sensors were mounted at or near the vertex of a play gym (here, 38×38×35 in) to locate the highest detectable entity in prone position. For infants in this Example, who could not move out of prone or reach above shoulder level in prone, the head was the highest entity measured by the sensors. The sensors recorded the head's distance from the floor and sent this information to the microcontroller. The microcontroller compared infants head height to its preset parameter or height of the threshold and activated the toy if conditions were met. The play center was set at an interval length of 10 seconds, which means the toy activated when the infant's head height was at or above the threshold and turned off when the head was lowered or after the preset interval length (10 s) was reached, even if the infant's head was above the threshold.

The associative learning testing protocol included two consecutive days of data collection. Day 1 consisted of 1) Pre-Baseline phase (30 seconds) 2) Baseline phase (2 minutes) 3) Acquisition phase (AQ_1,2,3 and 4 each 2 minutes in length). Day 2 was identical with the exception of the pre-baseline phase being excluded from the protocol. Parents were asked to limit interactions with their infant during the testing to allow the infant to focus on the learning task. All parents were advised to be in view of the infant and smile if the infant gets fussy. If the infant continued to be distressed, parents were asked to say “I am right here, you are doing okay” in an encouraging tone.

The pre-baseline phase, which is not a part of the typical mobile paradigm, was included in the testing to calculate the average height to which the infant was able to lift the head. This height was used as the individualized threshold to activate the toy during the rest of the paradigm. During the pre-baseline phase, infants were positioned in prone in the play center for 30 seconds. The position of an infant in the play center was standardized to ensure that the vertex of the infant's head was aligned at the center of the area covered by the play center sensors. A floor mirror toy was placed at the edge of the mat, in front of the infant for motivation. At the end of the 30 second pre-baseline phase, the play center calculated the Average head lift height (AHH). The AHH was used to set the Threshold head lift height (TH). TH is the height required by the infant to raise his/her head to activate the toy and was used for further testing. It was found that compared to thresholds that are above or below the average head lift height, infants reactivated the toy more often in the PPAC when the threshold equals the infant's average head lift height.

In the Baseline phase, infants were positioned in prone in the instrumented play center for 2 minutes. The play center toy did not activate in response to infants' movements in any way during this period. The purpose of the baseline phase was to provide us with information about the infants' head lift height when not in association with the activation of the play center toy.

In the acquisition phase, infants were in prone in the play center for 4 blocks of 2 minutes each. At the end of each 2-minute trial, the infant was rolled to a supine position for a 15-second break period. In the Acquisition phase, the play center toy was activated in response to infants' raising his/her head to a height equivalent to or greater than the TH. The toy remained on for a maximum of 10 seconds or turned off anytime the infants' head was below the AT. That is, in this phase, the toy turned off when 1 of 2 conditions were met: when the infant's head dropped below the threshold, or when the infant's head remained up for more than 10 seconds. The purpose of the acquisition phase was to surround the infants with an environment of associative learning allowing them the opportunity to learn the association through trial and error.

Few infants could complete 8 consecutive minutes in prone without signs of fatigue. Therefore, at the end of the each 2 minute trial infants were rolled to a supine position for a 15 seconds break period. Data collection was paused any time during the testing an infant cried continuously for 30 seconds or more. Each phase was re-attempted once if paused due to behavioral state. Data of infants who did not complete the baseline and first three acquisition trials on either day was excluded from the analysis, as the criteria developed to classify infants as short-term learners/nonlearners or retainers/nonretainers could not be applied.

The feedback devices included a choice among a roll-and-glow monkey toy, dance-and-move bug toy, and a mobile. All toys had similar features of glowing, singing, and moving.

Before the assessment, parents were asked if their infant would prefer any of these toys in particular to avoid infants losing interest in the toy during the assessment. Once the parent selected the toy for use with their infant, the toy was not changed within or across the 2 days of data collection. This was to ensure that infants received a consistent experience of the association between head lift and toy activation on day 1 and day 2 of testing.

### Data Processing

A computer was connected to the play center to record the following real-time data for each 2-minute block of testing (1 baseline and 4 acquisition blocks) at a sampling rate of 12 Hz: (1) infant's head lift height (in) and (2) toy status as on or off. Matlab was used to compute duration of head-above-threshold and toy on or “duration” (s), and frequency of toy reactivations or “reactivations” at the end of baseline and each acquisition phase. The duration variable was the total amount of time the infant's head was above the threshold and the toy was on, with a maximum of 10 seconds per episode of infant's head above the threshold. Reactivations are different from the number of times infants raised the head above the threshold or activated the toy. This variable accounts only for the number of times the toy was reactivated after a continuous activation of 10 seconds by the infant.

Using the standard learning and retention criteria from studies based on the Rovee-Collier mobile paradigm, a criterion was set a priori to categorize infants as short-term learners and retainers of the association learned on day 1. Infants were categorized as short-term learners if their duration (s) or reactivations in any 2 consecutive phases (2 min each) of the final 3 acquisition phases were 1.5 times higher than the duration or reactivations of the baseline on day 1. Retention of the association learned on day 1 occurred if duration or reactivations in any 2 consecutive phases of the final 3 acquisition phases on the second day of testing was 1.5 times higher than day 1 baseline. Although none of the criteria used data from the baseline phase on the second day of testing, this phase was included on day 2 of testing to allow application of the learning criteria on the second day if infants did not learn on day 1 of testing but needed an extra day of practice to discover the association in prone.

### Data Analysis

Descriptive statistics were used to describe the sample. In order to determine if 3-6 month old infants demonstrated associative learning, the percentage of infants who were categorized as short term learners was calculated from the processed data from day 1 of testing. In addition, in order to determine if 3-6 month old infants demonstrated retention of associative learning, data from the infants who learned on day 1 was used to identify percentage of infants demonstrating retention of the association, 24 hours later.

To further understand associative learning in prone, a post hoc analysis plan was developed to (1) assess if short-term learners met the learning criteria above the level of chance and (2) evaluate when in the protocol infants began to demonstrate short term learning. Descriptive statistics were used to describe differences between the 1) short term learners and non-learners and 2) retainers of the association learned and non-retainers. Both the reactivations and duration (FTR and DTO respectively) datasets were tested for normality. Because the FTR data had a Poisson distribution, a non-parametric Friedman test was used to evaluate phase differences in the FTRs within the short term learners. Wilcoxon rank-sum was used to compare differences in reactivations between the short term learners and non-learners. Since the DTO data was normally distributed, a Repeated Measure Analysis of Variance (RMANOVA) was used to compare the effects of phase of testing (Baseline, Acq 1, Acq 2, Acq 3 and Acq 4) on the DTO among the short term learners. An independent t test was used to compare differences in the duration between the short term learners and non-learners of the association in prone. An independent t test was used to compare differences in the duration between the short-term learners and nonlearners of the association in prone. Wilcoxon rank-sum test was used to compare the baseline reactivations on day 1 and day 2 of testing of learners, non-learners, retainers and non-retainers. Paired sample t test was used to compare the baseline DTO on day 1 and day 2 of testing of learners, non-learners, retainers and non-retainers. All the exploratory factors were described. Statistical analyses were completed using SPSS version 24 with alpha level set at 0.05 for the test statistics values and adjusted using a Bonferroni correction (adjusted α=0.0125).

### Results

The average age of the twenty-eight infants who participated in this study was 5 (0.9) months. The sample was 54% girls, 85% White and 100% of not Hispanic origin. A total of 6 infants' data was not included in the analysis; in 3 infants testing was stopped due to crying for more than 30 seconds continuously during the baseline, 2 infants often moved out of the play center sensor's coverage area by scooting backwards in prone and 1 infant's data file was corrupted and could not be processed further for analysis leaving an analyzable sample of 22 infants whose data were included in this analysis.

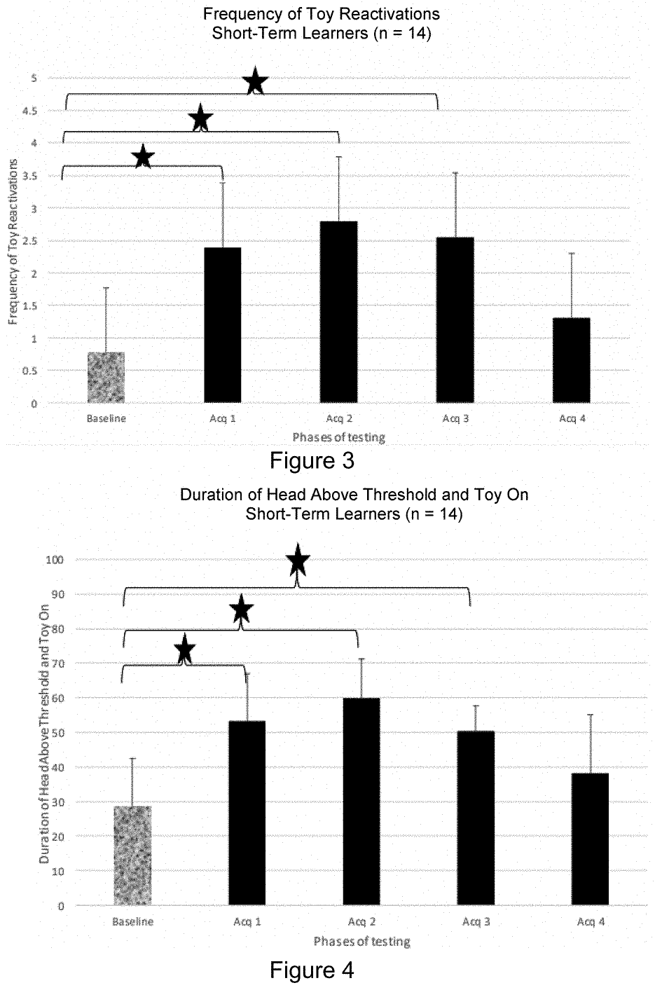

Fifty percent (n=14) of the infants who participated met the learning criteria on day 1 and were categorized as short-term learners of the association in prone position (FIGS. 2 and 3). Of the 14 short-term learners, 9 infants met the criteria based on their reactivations and duration, 3 infants based on their reactivations, and 2 infants based on their duration. Two of 8 infants (25% of those tested), who did not show learning on day 1, demonstrated short-term learning on day 2.

Of the 14 infants who demonstrated short-term learning on day 1 and should have been assessed for retention on the second day of testing, 1 infant was sick and 1 infant missed the visit. Thus, 12 short-term learners (from day 1) were assessed for retention. Three infants did not complete the acquisition phase due to excessive crying in the third acquisition phase of testing so their data could not be included. Three of the 12 (25%) short-term learners demonstrated a 1.5 times increase in their duration or reactivations of 3 consecutive acquisition phases on the second day than the first day baseline phase.

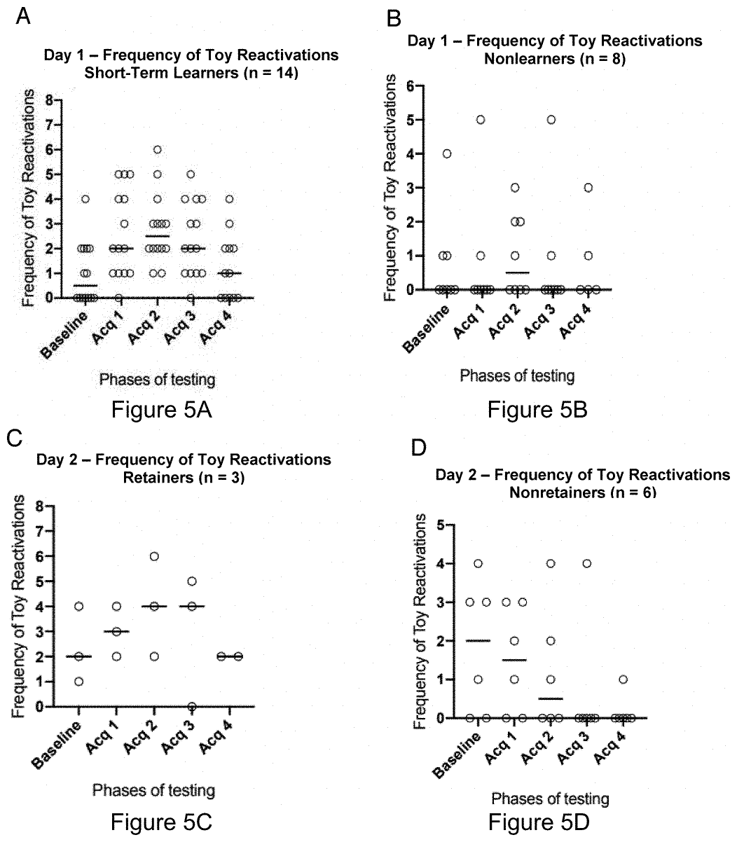

Frequency of toy reactivations is shown in FIG. 5A for short-term learners, FIG. 5B for nonlearners, FIG. 5C for retainers, and FIG. 5D for nonretainers. The horizontal lines represent the medians.

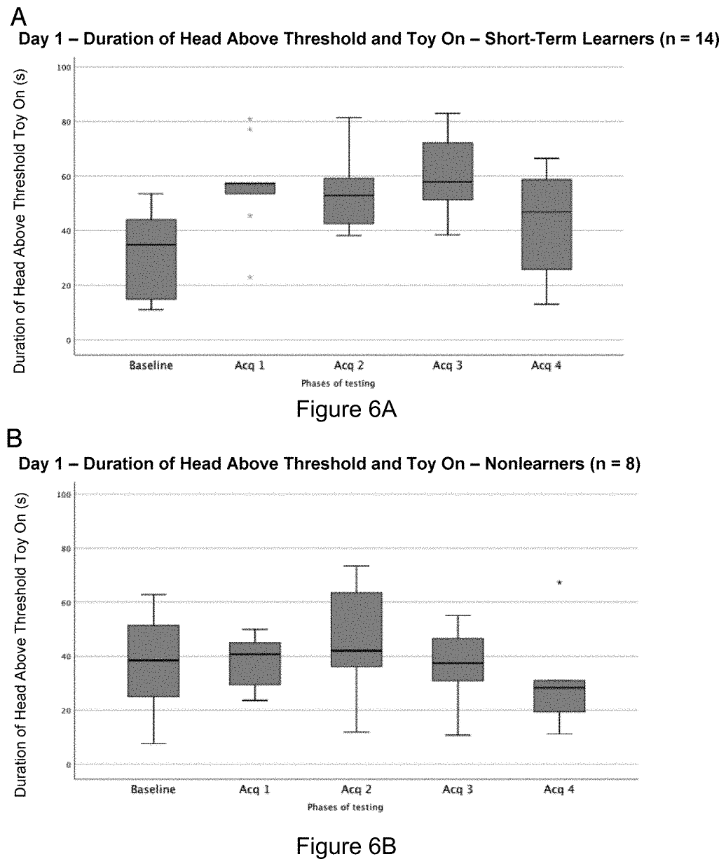

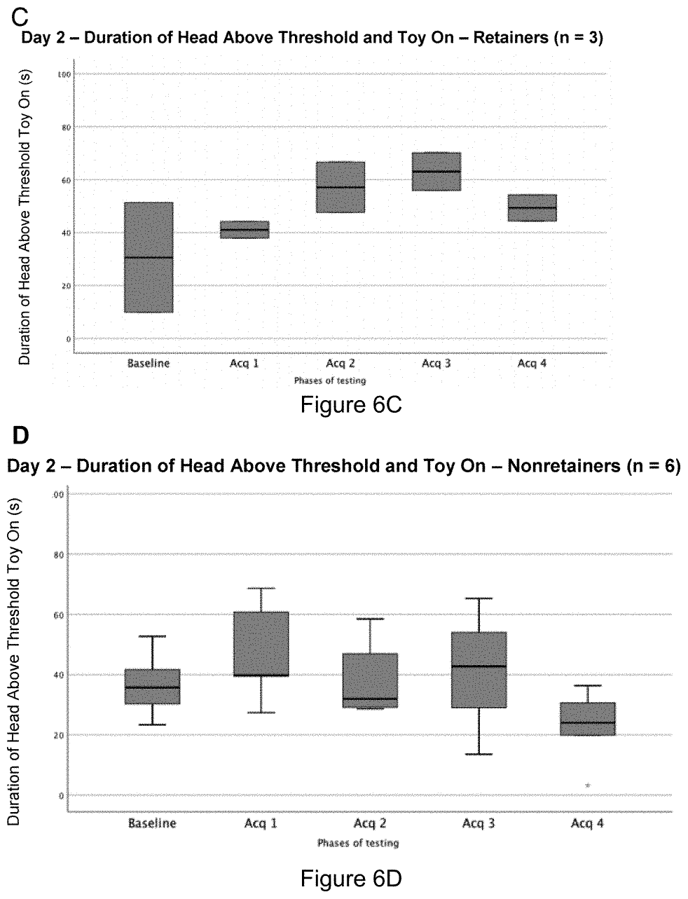

Duration of head above threshold and toy activation is shown in FIG. 6A for short-term learners, FIG. 6B for nonlearners, FIG. 6C for retainers, and FIG. 6D for nonretainers. Legends represent phases of testing, and number of infants tested in each phases is in parentheses. The horizontal line inside each boxplot represents median.

Numerical data corresponding with FIGS. 5A-5D and 6A-6D are presented in Tables 2 and 3.

TABLE 2
Descriptive statistics of Frequency of toy reactivations in short term learners and non learners

| | Baseline | | Acquisition 1 | | Acquisition 2 | | Acquisition 3 | | Acquisition 4 | |
|---|---|---|---|---|---|---|---|---|---|---|
| Day 1 testing | M (SD) | | M (SD) | | M (SD) | | M (SD) | | M (SD) | |
| Short term | 0.76 | (.92) | 2.38 | (1.66) | 2.78 | (1.42) | 2.53 | (1.39) | 1.3 | (1.42) |
| learners (n = 14) | | | | | | | | | | |
| Non-learners | 0.75 | (1.38) | 0.75 | (1.75) | 1.0 | (1.19) | 0.75 | (1.75) | 0.8 | (1.30) |
| (n = 8) | | | | | | | | | | |

Duration of toy ON (in seconds) in short term learners and non learners

| | | | | | | | | | | |
|---|---|---|---|---|---|---|---|---|---|---|
| Short term | 28.59 | (13.85) | 53.13 | (13.80) | 59.71 | (11.48) | 50.21 | (7.46) | 38.01 | (17.06) |
| learners (n = 14) | | | | | | | | | | |
| Non-learners | 37.06 | (21.71) | 39.36 | (11.38) | 45.68 | (21.60) | 36.10 | (16.88) | 32.61 | (30.29) |
| (n = 8) | | | | | | | | | | |

TABLE 3
Descriptive statistics of Frequency of toy reactivations in retainers vs non retainers

| | | | Acquisition | | Acquisition | | Acquisition | | Acquisition | |
|---|---|---|---|---|---|---|---|---|---|---|
| Day 2 | Baseline_FTR | | 1_FTR | | 2_FTR | | 3_FTR | | 4_FTR | |
| testing | M(SD) | | M(SD) | | M(SD) | | M(SD) | | M(SD) | |
| Retainers (n = 3) | 1.5 | (0.83) | 3.0 | (1.0) | 4.0 | (2.16) | 3.0 | (2.14) | 2.0 | (1.90) |
| Non-retainers | 1.83 | (1.72) | 1.5 | (1.37) | 1.16 | (1.6) | 0.6 | (1.63) | 0.16 | (0.40) |
| (n = 6) | | | | | | | | | | |

Duration of toy ON (in seconds) in retainers vs non- retainers

| | | | | | | | | | | |
|---|---|---|---|---|---|---|---|---|---|---|
| Retainers (n = 3) | 32.44 | (20.27) | 50.62 | (6.25) | 52.98 | (11.25) | 47.04 | (10.02) | 54.23 | (21.64) |
| Non-retainers | 32.89 | (6.77) | 49.31 | (19.37) | 34.58 | (13.57) | 34.93 | (23.32) | 23.56 | (14.47) |
| (n = 6) | | | | | | | | | | |

Phase Comparison within Short Term Learners and Non-Learners

Findings from the Friedman test suggested a significant difference in the reactivations of short term learners within the 5 phases of testing (χ²=21.48, P=0.003).Short-term learners increased their reactivations significantly from baseline to Acq 1 (χ²=−1.90,P =0.007), Acq 2 (χ²=−2.3, P=0.001) and Acq 3(χ²=−2.25, P=0.001). No significant difference was seen between baseline and Acq 4 (χ²=−0.50, P=0.48) for the short-term learners (FIG. 4A). RMANOVA results indicated a significant difference in the duration among the short-term learners within the 5 phases of testing [F(4,7)=7.19, P=0.000]. Pairwise comparisons revealed that short-term learners had a significant increase in the duration from baseline by an average of 24.5 seconds in Acq 1 (P=0.027), 31.1 seconds in Acq 2 (P=0.016), and 21.6 seconds in Acq 3 (P=0.001). After Bonferonni correction, only the difference in the duration between baseline to Acq 3 survived significance. No significant difference in the mean duration was seen between baseline and Acq 4 (P=1.0) for the short-term learners (FIG. 4B). In contrast to the short-term learners, no significant differences were seen in the reactivations (χ²=0.364, P=0.98) and duration [F(1,4)=1.06, P=0.40] within the baseline and acquisition phases of testing among the nonlearners on day 1 of testing.

### Phase Comparisons Between the Short Term Learners and Non-Learners

A direct comparison of the learners and nonlearners reactivations and duration was used to quantify difference in the performance pattern during the testing protocol. The Wilcoxon rank-sum test indicated a significant difference in the reactivations between the short-term learners and nonlearners at Acq 1 (z=−2.62, P=0.008), Acq 2 (z=−2.59, P=0.01) and Acq 3 (z=−2.55, P=0.01). No significant group difference was found in reactivations at baseline (z=˜0.67, P=0.57), and Acq 4 (z=−0.83, P=0.40). For the duration comparisons, t-test statistics indicated a significant difference at Acq 1 [t(20)=−2.86, P=0.00], Acq 3 =[t(20)=−3.12, P=0.00]. No significant differences between the groups were seen at baseline [t(20)=1.78, P=0.08], Acq 2 [t(20)=−0.89, P=0.38] and Acq 4=[t(14)=−0.94, P=0.36].

### Day 1 Vs Day 2 Baseline FTRs and DTO Comparisons—Evaluation of Retention of Learning During Day 2 Baseline

No significant difference was found between the baseline FTRs on day 1 and day 2 of short term learners (Z=1.20, p=0.22) and non-learners on day 1 of testing (Z=1.50, p=0.414). Similar results were seen when the day 1 and day 2 baseline DTO of learners [t(11)=0.805, p=0.44] and non-learners [t(4)=0.950, p =0.39] were compared against each other. However, 40% of the short term learners had a 1.5 times higher FTRs on the 2^{nd} day's baseline compared to day 1's baseline.

No statistical significant difference was seen in the day 1 and day 2 baseline's FTRs (Z=1.84, p=0.06) and DTO [t(5)−=0.50, p=0.64] of short term learners who did not retain the association a day later (non retainers). However, 60% of the non-retainers had a 1.5 times higher FTRs on 2^{nd} day's baseline compared to day 1's baseline. Similarly, no significant difference was seen in the day 1 and day 2 baseline's FTRs (Z=1.34, p=0.18) and DTOs [t(2)=−0.1.91, p=0.19] of infants who retained the association a day later.

### Exploratory Factors Impacting Learning

A 2 tailed independent t test showed that infants' age (p=0.61) and AIMS prone score (p=0.19) did not differ between the groups. However, a higher percentage of short term learners completed the testing compared to the non-learners (Table 4).

TABLE 4
Exploratory factors impacting learning

| | Short term | |
|---|---|---|
| | Learners | Non-learners |
| Age in months, mean (SD) | 4.9 (.75) | 5.10 (1.08) |
| AIMS prone skills score, mean (SD) | 7.21 (1.12) | 6.5 (1.3) |
| Percentage of infants who completed the last | 79 | 62 |
| block of testing in the acquisition phase (%) | | |

### Discussion

At the outset of this Example, it was expected that infants who participated would demonstrate short-term learning of an association between their head lifts with the activation of a toy and retention of the association learned on day 1, 24 hours later. Findings from the first day of testing supported the expectation. It is encouraging that half of the sample demonstrated associative learning, which is challenging both motorically (lifting head in prone) and as a novel learning task.

Contrary to the expectations, poor retention was found in infants who failed to learn the association in prone on day 1. A majority of the nonlearners on day 1 did not meet the learning criteria with an extra day of practice.

To understand factors that may support infants' ability to learn the association in prone, a post-hoc analysis was conducted to determine if there exist a difference in infant's age and Alberta Infant Motor Scale (AIMS) prone score between the short-term learners and nonlearners. A 2-tailed independent t test showed that infants' age (P=0.61) and AIMS prone score (P=0.19) did not differ between the groups (Table 4). Infants' tolerance for prone position may support their ability to complete the testing and provide additional opportunities to learn the association between their upper body movements and the activation of a toy. Although infants were screened for their tolerance in prone to determine eligibility, infants were still included who clearly “liked” tummy time as well as infants who passed the screening but were tired after 5 or 6 minutes into the testing. This may explain a higher percent of short-term learners (79%) completing the last acquisition block of testing compared to the nonlearners (62%). In addition to this, the possibility of differences in individual infant's cognitive abilities such as capacity to problem solve, attend to the play center toy may have played a role in an infant's ability to learn or not learn the association.

The learning criteria present a unique way to explore the learning strategies infants may have used to discover the activation of the toy. An infant with high reactivations may have also kept the toy on for a long period of time due to the 10 seconds of continuous activation required to receive credit for frequency of toy reactivations. However, the reverse may not always be true, because an infant can cross the threshold many times to keep the toy on without holding the head above the threshold for a maximum of 10 seconds. The finding that a majority of the short-term learners had a 1.5 times increase in their reactivations and duration in the acquisition phase of testing compared with the same day's baseline suggests that in the acquisition phase the short-term learners raised their head in prone at or above the threshold to activate the toy, held that position for 10 seconds and then crossed the threshold again to reactivate the toy. In contrast, those who did not learn demonstrated a variety of patterns in the acquisition phase of testing: (1) crossing the threshold many times with a burst of head lifts that were less than 10 seconds above the threshold (nonlearners=19.42±3.98; short-term learners=15.12±1.49) and (2) not achieving the threshold to activate the toy (nonlearners: average threshold height=9.14±1.34 in, average head height=9±0.53 in; short-term learners: average threshold height=9.96±1.46 in, and average head height=10.16±0.33 in). In these cases, the reactivations and duration were low because the infant did not learn the association to turn the toy on for the required time limit and the need to reactivate with a lowering and re-lifting of the head.

A majority of infants did not meet the criteria to be identified as retainers of the association between their movements and the activation of the toy, 24 hours later. Possible explanations of the discrepancy found in the retention rate of infants among different paradigms could be: (1) the use of a baseline phase on day 2 may have caused a “wash out” of the association learned on day 1 and interfered with retention, and (2) the modified mobile paradigm and this Example's paradigm in prone being more challenging to learn and retain than the traditional mobile paradigm. Due to the novel nature of the instant paradigm, the testing procedures established by Rovee Collier and colleagues in their work on infant learning and memory were followed. Though studies based on the Rovee Collier mobile paradigm have a baseline phase on the second day, there exists an inconsistency in the interpretation of the second day's baseline data. In this Example, the assessment of learning in infants was prioritized by including a baseline phase on day 2 to see if infants needed an extra day of practice to discover the association in prone position. The inclusion of baseline phase on day 2 allowed for applying the short-term learning criteria, but it might have interfered with retention.

Another potential limitation of this Example was the high dropout rate (50%, day 1 and day 2 combined), which was due primarily to infants' intolerance for prone position, wear and tear of the prototype play center, or loss of infant's interest in the play center toy during the assessments. No descriptive differences were found in the demographic characteristics between the infants who dropped out and infants who were retained. Of the 28 infants recruited, it was necessary to stop data collection for 9 infants as 6 infants cried excessively during the testing and for 3 infants the prototype play center did not function properly. The majority of infants who dropped out did so due to excessive crying during the baseline phase or third acquisition phase of testing. In both the situations, the learning and retention criteria could not be applied, which caused exclusion of the data. A high number (75%) of 3 to 6 month-old infants' exhibited intolerance for prone position during the screening. These issues can be solved by changing the eligibility criteria to infants who can tolerate 10 minutes of prone play and improving the efficiency of the technology by adapting the prototype. For infants who do not tolerate prone position, it might be beneficial to configure a play center to provide continuous feedback to infant's head raise in prone as a tool to encourage prone play in infants' routine. This Example's small sample size limited the ability to do a detailed analysis of the factors such as infants' age, tolerance for prone, motor skills in prone, problem solving abilities that may predict short-term learning of an association in prone position in infants.

### Clinical Relevance

This Example provides two avenues for physical therapists to contemplate in their practice: 1) The play center and an associative learning paradigm may have the potential to detect early motor learning delays in infants or the impact of atypical motor control on early learning; and 2) The ability to measure learning using this prone associative learning paradigm may lead to the developmental of associative learning based intervention to train infants to modify their motor control in prone. The temporal and spatial features of the task provides an infant with opportunities to learn the anticipatory and predictive piece of skill development consistent with the interplay between motor and cognitive skills in early learning.

### Example 2

### The Feasibility of a Clinical Trial to Compare High and Low Tech Interventions to Enhance Motor Development and Prone Tolerance in 3-6 Months Old Infants

The purpose of Example 2 was to evaluate the feasibility of conducting a randomized clinical trial to assess the effectiveness of a home based intervention using positive reinforcement strategies to improve tolerance for prone positioning and positively impact motor development in 3-6 months old infants. Specifically, the aims of this Example were to determine the feasibility of delivering the proposed interventions and evaluate if the proposed outcome measures are able to detect change in motor skills and prone tolerance in infants. The secondary aim was to describe which factor(s), in addition to the key elements of the interventions, may influence prone motor skills during the intervention period.

### Methods

### Participants and Setting

A convenience sample of ten 3 to 6 month old infants born at term (50% female; mean age=4.19 months, SD=0.7 months) participated in this parallel group randomized feasibility trial. Infants were identified from the community and parents provided informed consent.

Full term infants with poor prone motor skills and poor prone tolerance were eligible for the study. Poor prone motor skills were defined as a score of 2 to 6 in the prone subsection of Alberta Infant Motor Scale (AIMS). Poor prone tolerance was identified as fussing/crying for more than 30 seconds during a five minute period in prone, and validated by parents' statement that the infant did not enjoy prone play. Fussing and crying was defined using the descriptors from Brazelton infant behavioral state (state 6) (Brazelton 1918-TB. Neonatal Behavioral Assessment Scale. 3rd ed. (Nugent J K, ed.). London: Mac Keith Press: Distributed by Cambridge University Press; 1995). Infants born with brain injury or any neurological event associated with a risk of neurodevelopmental disabilities, musculoskeletal deformity, genetic syndromes, visual and hearing problems, or any other disorders or medical complications limiting participation in assessments and intervention were excluded.

All infants enrolled in the study participated in the same assessment schedule, regardless of the group assignment. Post baseline assessment and collection of demographics and socio-economic status information, infants were randomized to the High-technology group (HTG) or the Low-technology, Education group (EG). All assessments sessions were conducted either in the infant's home or at a university lab based on parent's choice. Visits were scheduled during a time of the day when the infant was awake and playful.

### Intervention

Infants in the HTG participated in a 3 week, home based intervention program led by parents to improve their infant's prone tolerance and promote motor development. The intervention included 1) positively reinforcing infant's initial efforts to lift their head in prone and 2) progressing the intervention to provide the “just right” challenge for prone play daily. The “just right” challenge involved adjusting the threshold for activation of positive reinforcement to push the infant to progress towards higher prone motor skills over multiple sessions. The intervention was administered by the use of a play center generally consistent with FIGS. 1A and 1B, with the exception that the toy was not attached to a support but rather was placed adjacent to the mat. Briefly, the experimental play center was a high technology device that included 1) ultrasonic sensors 2) a microcontroller (here, an Arduino Uno), and 3) dancing/singing toy. The sensors located the position of infants' head in space and records the heads' distance from the floor. The microcontroller compared infants head height to its preset parameter or height of the threshold and activated the toy if conditions were met.

On day 1 of the intervention the parent was educated on the importance of prone play with the use of a “Back to sleep, Tummy to Play” brochure from the American Academy of Pediatrics (AAP). This was followed by a demonstration of the features and functions of the play center. The parents were coached to set the play center to reinforce movements at their infants' “just right” challenge of prone play, as defined below. Parents were oriented to the intervention model so they understood when their infant lifts his/her head over the established threshold the toy will activate and sing and dance until the infant's head goes below the threshold.

A four step coaching model was used to support parent's ability to understand the concept of “just right” challenge of prone play:

Step 1: Three “just right” challenge levels of prone play (easy, moderate and challenging) were determined using infant's average head lift height (AHH) calculated on the first day of intervention. Based on pilot data, the average height was used to calculate the “just right” prone play activity levels: a) “Easy” level—threshold height is set at 25% below the day 1 average head lift height b) “Moderate”—threshold height equals the day 1 average head lift c) “Challenging” level—threshold height is 25% above the day 1 average head lift height.

Step 2: The easy, moderate and challenging threshold heights were written in an intervention manual for parents to refer during the intervention period. All parents were asked to begin the intervention from the “Easy” prone play activity level. It is important that infant's first experiences using the play center allowed them the opportunity to activate the toy multiple times, so the infant learns the association between their movement and the toy activation. The moderate and challenging levels were used to continue to positively reinforce the head lift as the infant becomes stronger and more tolerant of prone.

Step 3: Parents were asked to administer at least 30 minutes of prone play with the play center on 15 days over a span of 3 weeks. Parents had 24 hours access to the play center in the home and could pace the intervention in 4 to 5 short periods (6-8 minutes) to avoid fatigue and gradually increase the duration based on their infant's behavioral state.

Step 4: Parents were coached to advance to the next “just right” challenge level of prone play when their infant's performance met the increment criteria. The increment criteria was “if the infant is able to complete at least 30 minutes of prone play in the play center without crying, and activating the toy at least once”. If the parent perceived their infant met the increment criteria they advance to the next level (moderate or challenging) based on the threshold levels provided by the interventionist at the first visit. Along with the play center, parents received an intervention manual and an activity log, both paper and electronic versions. The manual included an orientation to the play center and how to adjust the “just right” challenge prone play level. The manual also included the infant's individually determined “Just Right” Challenge thresholds as an integer so the parent could adjust the threshold knob as directed (Step 2). The activity log was designed to capture the amount of times parents report the infant played in prone and will be described further in the assessment section. Researcher met the parent on Day 7 and Day 14 of the intervention period to discuss any issues with play center, talk about the intervention, and ask parents to demonstrate the intervention and how they determined the “just right challenge” level for that day's practice session.

Parents of infants in the EG received the same “Back to sleep, Tummy to Play” brochure as the HTG. Parents were asked to incorporate at least 30 minutes of prone play in their infant's daily routine, 15 days over 3 weeks. Parents were advised to pace the intervention in 4 to 5 short periods (6-8 minutes) to avoid fatigue and gradually increase the duration based on their infant's behavioral state. Using the brochure as a guide parents were provided with tips to encourage prone play including by placing themselves or toys in front of the infant or holding the infant on their chest and talking to the infant. Use a towel roll or u-shaped pillow under the infant' chest was described and demonstrated. On day 1 of the intervention, parents received an intervention manual outlining the goal of 30 minutes of prone per day and an activity log with the same questions as the HTG to document time in prone. Researcher met the parent on Day 7 and Day 14 of the intervention period to discuss their infant's prone play routine and any issues encountered in administering prone play or completing the activity log.

### Assessments

To assess feasibility of completing a clinical trial of the proposed High technology intervention to advance prone motor skills, enrollment and outcome assessment completion statistics were evaluated. To estimate the enrollment and retention rate, number of parents who expressed interest in the study through a phone call or email to the research team, number screened for eligibility, enrolled, and retained were tracked.

Adherence of parents and interventionist to the HTG and EG interventions was evaluated by tracking the frequency of intervention visits completed and key intervention principles administered by the interventionist and parents. After each intervention visit, the interventionist recorded the principles utilized during the session to self-assess her adherence to the intervention procedures. In addition, the interventionist repeated this self-assessment a week later to ensure reliable measures of adherence. To assess difference in the HTG and EG, information from the activity log was used to document the number of session parents used the play center (HTG only), amount of time per day infants' spent in prone, and amount of time per day the parent-infant dyad spent in face to face interaction in prone. A video of the Day 7 and Day 14 intervention session was used to evaluate parents' adherence to the interventions. Videos were scored by the interventionist using a similar intervention principles checklist used to score interventionist's adherence. These values were compared with the anticipated feasibility thresholds (Data analysis section) to determine parent's adherence in both the interventions.

For the identification of change in infants' tolerance to prone position we developed a measure of Prone Tolerance. Infants were placed in prone position by the researcher for a maximum 15 minutes to assess prone tolerance. The testing began as soon as the examiners hands left the infants body and ended after 15 minutes or stopped any time the infant cried for more than 30 seconds. Crying was defined using the descriptors from Brazelton infant behavioral state (State 6). (Brazelton 1918—TB. Neonatal Behavioral Assessment Scale. 3rd ed. (Nugent J K, ed.). London: Mac Keith Press; Distributed by Cambridge University Press; 1995.) The time lapse between the start and end of the trial was calculated as a measure of the infant's tolerance towards prone position. The score ranges from 0.5-15 minutes, where a score of 0.5 represents that the infant cried for 30 seconds immediately after being placed in prone position and a score of 15 represents the infant did not cry for more than 30 seconds during the 15 minutes of testing. This measure has not been validated yet.

AIMS and Gross Motor Function Measure (GMFM)-66 were completed at baseline and end of the intervention (EOI) period (Day 0 and Day 22). These measures were assessed for feasibility and sensitivity to determine which will be used in future studies. While the measures were administered by the same person who completed the intervention, a blinded, and reliable assessor scored the video tapes of the assessments to ensure objectivity. The AIMS is a reliable and valid observational assessment scale used to measure gross motor abilities in infants from birth through independent walking. It consists of 58 items organized into four positions: 21 prone items, 9 supine items, 12 sitting items and 16 standing items. Each item is scored as either “observed” or “not observed”. The “least mature” and “most mature” item observed is marked for each of the four position. The items observed between the least mature and most mature item in a position represents the “window” of current skills for that position. The AIMS raw score for each position is the credit infant receives for sum of all the items before the window and for each items observed with in the window. The sum of the raw score in each position is the total AIMS score. AIMS evaluates three aspects of motor performance—weight-bearing, posture and anti-gravity movements. It can be completed within 15-30 minutes.

The Smallest Detectable Change (SDC) for AIMS is 3.88 raw score points. The GMFM-66 is a valid and reliable clinical measure designed to evaluate changes in gross motor function in children of 0-5 years of age with cerebral palsy (CP). While the validity and reliability of GMFM has been evaluated for children with CP and Down syndrome, Example 2 uses it for typically developing children as: 1) GMFM-66 does a detailed sampling of motor skills that are “typical” of normal development and 2) some use cases may include children who are at high risk of cerebral palsy, making it an appropriate measure for this feasibility trial. GMFM-66 consists of 66 items under 5 dimensions: Lying and Rolling, Sitting, Crawling & Kneeling, Standing and Walking, Running and Jumping. It uses a 4 point scoring system to score each item on the scale of 0-3 where (0=does not initiates, 1=initiates, 2=partially completes and 3=completes). The sum of the score on each item is the total GMFM score and represents the percent of the test items the child could complete. The SDC for GMFM-66 is 3.71 points in children with a mean age of 3.7 years. Percentage of infants who reached SDC was calculated to compare the sensitivity of the AIMS and GMFM-66. A standardized set of toys were used for both AIMS and GMFM to motivate infants to demonstrate a particular skill, if the skill was not observed during free play.

### Assessment of Potential Mediators

To document that parents were completing the intervention as planned during the non-supervised sessions, it was determined how much time the infants spent in prone each day. Parents selected one of the following options on the activity log to report the amount of time the infant was in prone every day: <15 minutes, 15-30 minutes and >30 minutes of prone time. This information was used to determine the percent of days the total sample practiced prone for <15 minutes, 15 to 30 minutes or greater than 30 minutes out of the total expected parent reports (5 reports per week for each participant; for HTG (4×5) 20 and for EG (5×5) 25 total parent reports). If the log was not completed or no record of time spent in prone was made in the activity log, no prone time was assumed for that day and a duration of 0 was included in calculations for that day. To determine if infants in the HTG progressed through the “Just right” levels of prone play, each week's prone play level (easy, moderate and challenging) was tracked using the activity log and parent reports during the weekly visits.

### Data Analysis

Descriptive statistics were used to describe the study sample and feasibility thresholds were determined a priori. Enrollment and retention were considered feasible if 75% of the eligible infants are enrolled and 90% are retained. The intervention was considered feasible if the interventionist reviews 90% of the key principles of the intervention with the parents, parent completes 30 minutes of prone play on at least 85% of planned session (in the play center for the HTG), and parents correctly sets the “Just Right” level of prone play 100% of the time. We considered the prone tolerance measure to be feasible if more than 90% of the infants completed the measure without achieving the lowest or highest score. To determine if AIMS and GMFM-66 are sensitive to change over time, we compared the percent of infants from the total sample whose change on the AIMS and GMFM-66 from baseline to end of intervention reached the SDC. In addition, to evaluate if AIMS and GMFM-66 are sensitive to detect differences in the HTG and EG we calculated the percent of infants in each group who improved more than the SDC on each measure. Cohen's d effect sizes were also calculated with 95% confidence interval (CI) for the total sample and on the group differences in the AIMS, GMFM-66, and prone tolerance changes scores from baseline to end of intervention for use in planning for the future studies. Consistent with the CONSORT guidelines a formal sample size calculation was not performed for this feasibility study but the results of this study will allow for sample size calculations in future studies.

Given the nature of this Example as a feasibility trial the individual infant's age, AIMS, GMFM-66 and prone tolerance scores at baseline, weekly progression of the duration of prone play, change in the AIMS, GMFM-66 and prone tolerance scores at the end of the study are evaluated descriptively (Table 5). While the sample is too small for a full mediation evaluation, the descriptive were used to consider factors which may need to be evaluated in future research, consistent with the secondary aims of the study.

TABLE 5
Possible mediators leading to change in prone tolerance and motor development

| | Total Sample | | | | | | | | | | |
|---|---|---|---|---|---|---|---|---|---|---|---|
| | | Baseline motor development | | | Frequency of <15 min, 15-30 min and >30 | | | | | | Change |
| | | and prone tolerance scores | | | min of prone play sessions per wk | | | | | Change | in prone |
| High | | | | Prone | Time in | | | | Change | in GMFM- | tolerance |
| Tech | | AIMS | GMFM- | tolerance | prone | | | | in AIMS | 66 raw | score |
| group | Age | score | 66 score | score | (min) | Week 1 | Week 2 | Week 3 | raw score | score | (min) |
| Infant 1 | 3.46 | 13 | 21.2 | 2.5 | <15 | — | — | — | 6* | 5.5* | 11.1 |
| | | | | | 15-30 | 5 | — | — | | | |
| | | | | | >30- | — | — | — | | | |
| | | | | | No report | — | 5 | 5 | | | |
| Infant 2 | 3.82 | 15 | 23.4 | 1.9 | <15 | 1 | | 1 | 6* | 4.6* | 11.9 |
| | | | | | 15-30 | 2 | 1 | 2 | | | |
| | | | | | >30- | 1 | 4 | 2 | | | |
| | | | | | No report | 1 | — | — | | | |
| Infant 3 | 5.69 | 20 | 30 | 3.9 | <15 | — | — | — | 4* | 4.8* | 11.1 |
| | | | | | 15-30 | 1 | — | — | | | |
| | | | | | >30 | 2 | 5 | 5 | | | |
| | | | | | No report | 2 | — | — | | | |
| Infant 4 | 4.77 | 16 | 27.3 | 2.25 | <15 | 2 | 2 | 2 | 8* | 0.7 | 6.2 |
| | | | | | 15-30 | 3 | 2 | 2 | | | |
| | | | | | >30 | — | 1 | 1 | | | |
| | | | | | No report | — | — | — | | | |

### Results

Of the infants screened for eligibility, 76% infants were eligible for participation. The 24% not eligible were either not in the age range or had prone motor skills and prone tolerance above the required range for inclusion. All infants who met the eligibility criteria consented to participate in the study and completed the baseline testing, resulting in a sample of 10 infants, 5 in each group. All 5 infants in the EG completed the study. Of the 5 infants in the HTG, one infant was lost to follow up after the baseline testing. The parent of the infant who dropped from the study shared the concern of not being able to use the play center due to sibling interference. Parent and Interventionist Adherence

The interventionist completed 95% of the total required intervention session with 100% adherence to the key principles. One session was missed due to one infant dropping out of the study after the baseline visit.

High Technology Group—Of the anticipated 15 days of parent reported intervention, 96% of the time parents reported information on prone play in the activity log. One parent did not complete the log after a week in to the intervention as the parent returned to work and the infant spent most of the day in the daycare. Parents of infants in the HTG reported using the play center on 93% of the 15 anticipated intervention days. Only 30% of the 15 anticipated sessions, parents in the HTG group used the play center for >30 minutes per day during the study. The average duration of play center use per day was 18(7) minutes as reported by parents in the activity log (Table 6).

TABLE 6
High technology group intervention “active
ingredients” descriptors

| | Average play center | | | | | |
|---|---|---|---|---|---|---|
| | use (mins/day) | | | “Just Right” prone play level | | |
| HTG | Wk 1 | Wk 2 | Wk 3 | Wk 1 | Wk 2 | Wk 3 |
| Infant 1 | 11 | NR | NR | Easy | Moderate | Moderate |
| Infant 2 | 13 | 17 | 25 | Easy | Easy-Moderate | Moderate- |
| | | | | | | Challenging |
| Infant 3 | 12 | 34 | 30 | Easy | Easy-Moderate | Moderate- |
| | | | | | | Challenging |
| Infant 4 | 16 | 16 | 17 | Easy | Easy | Easy-moderate |

NR = no report.

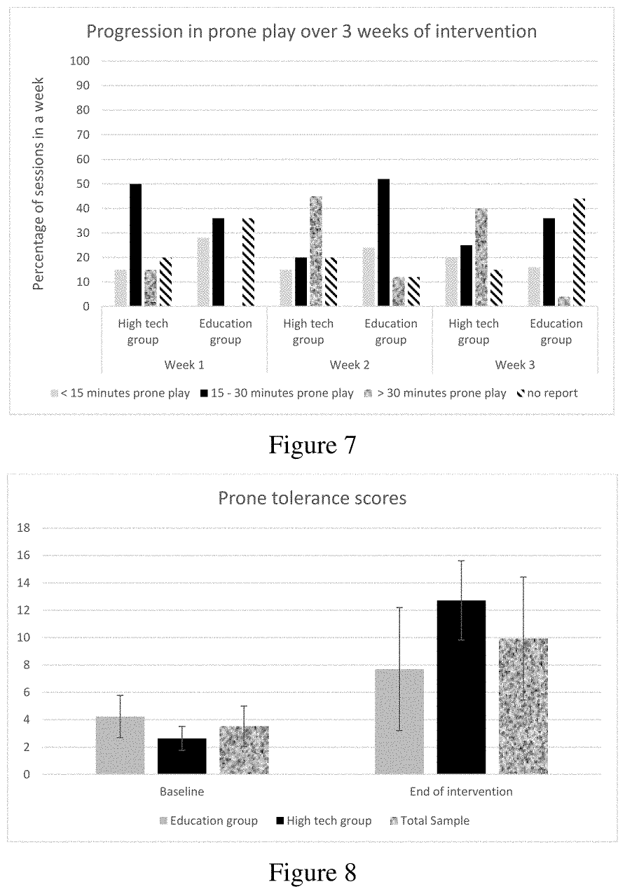

Of the anticipated 15 sessions of prone play, 93% of the time infants practiced prone play. Parents reported their infant practiced 15-30 minutes and >30 minutes of prone play 27% and 47% of the time respectively (Table 5, FIG. 7). Parents in the HTG demonstrated 100% adherence towards identifying the “Just Right” level of prone play by mentioning the increment criteria when asked about it during the weekly visits (Table 6). However, 75% of parents progressed their infant to a higher level than the one recommended. The parents reported that the suggested level was too easy for their infant. Parents often reported that completing 30 minutes in any prone play level is challenging due to infants' poor tolerance after a certain period of time. So they would increase the difficulty level to provide the infant with an opportunity to practice pushing up their upper body to a higher level until the infant fatigued and became intolerant of prone.

Education Group—Of the anticipated 15 days of parent reports, 69% of the time parents reported information on prone play in the activity log. Fifty three percent of the time infants practiced prone play. On 46% of the reported days the infant practiced prone play for 15-30 minutes and only 4% of the time for at least 30 minutes per day (Table 5, FIG. 7).

### Prone Tolerance and Motor Development

The prone tolerance measure developed for this example is a feasible measure. A high percent of infants (95%) completed the prone tolerance measure without achieving the lowest (0.5 minutes) or highest (15 minutes) score on the measure.

### Sensitivity of Measures to Change Over Time

Eighty eight % (total sample 8 of 9) of infants had a positive change in their prone tolerance score. While 100% and 88% (total sample 8 of 9) of infants had a positive change in their GMFM-66 and AIMS score respectively, only 44% of infants had a change in the AIMS more than its SDC and 44% had a change more than the GMFM-66's SDC. A Cohen's d of 1.91, 95% CI (0.72-2.92) for prone tolerance score, 0.77, 95% CI (−0.22, 1.69) for GMFM-66 score and 1.42, 95% CI (0.39-2.46) for AIMS score was found for the total sample from baseline to end of intervention (FIGS. 8-10).

### Sensitivity of Measures to Detect Group Differences

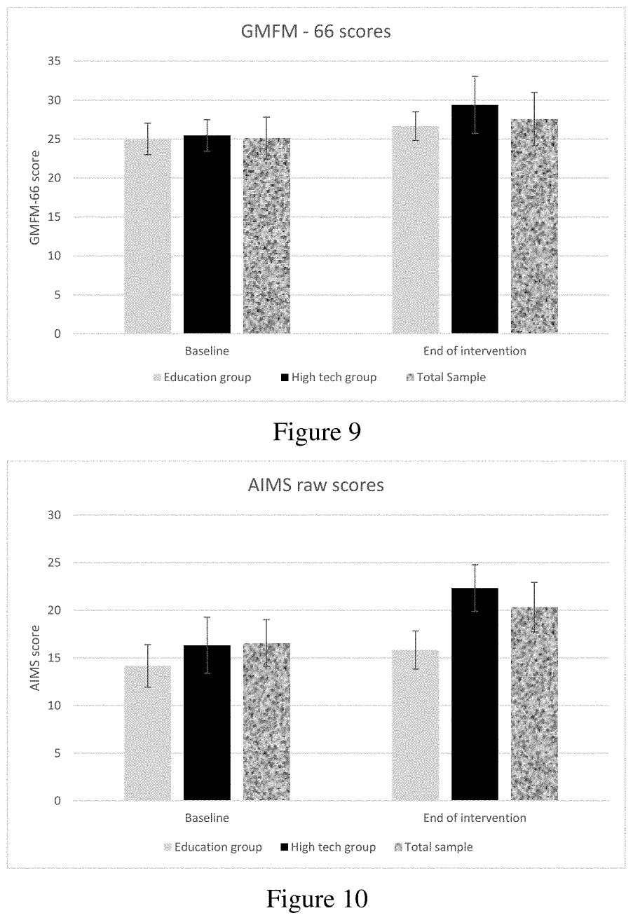

In order to measure the sensitivity of the outcome measures to different interventions group differences were calculated. This is not a measure of efficacy of the intervention. While prone tolerance increased in both groups the HTG increased a mean of 10.08 (2.5) minutes while the EG increased a mean of 3.46 (4.60) minutes (FIG. 8, Table 4). In the HTG and EG, 75% and 20% of infants respectively had a change in their GMFM-66 score more than the SDC (FIG. 9, Table 5). All infants in the HTG had a change in their AIMS score more than SDC; however, none of the infants in the EG had a change in their AIMS score that achieved the SDC. (FIG. 10 and Table 5). A Cohen's d of 1.71, 95% CI (0.03-3.01) for prone tolerance score, 0.86, 95% CI (−0.60, 2.13) for GMFM-66 score and 2.97, 95% CI (0.84-4.43) for AIMS score was found for the differences in the change scores between the groups.

### Description of Potential Mediators

There was a variability in the rate of progression in duration of prone play between and within groups. For example, infants 1, 2 and 3 appeared to make greater gains in all outcome measures than infant 4 (Table 5). However, infant 4 does not appear to have difference in the baseline scores and is of a similar age at baseline to those that improved the most. The only notable difference is that infant 4 did not progress in the time she spent in prone based on parent report as quickly as infants 2 and 3. Infant 9 in the EG had the lowest motor development scores at baseline and also made the least progress in the duration of prone play at home in the EG. However, infants with similar scores made improvements in the HTG. Thus, age does not appear to be related to outcome scores either.

### DISCUSSION AND CONCLUSION

The result of this example suggest that the use of a high tech intervention to enhance prone tolerance and motor development is feasible. Families were eager to participate as reflected in the 100% enrollment and 90% retention rate. The only parent who opted out of participation from the HTG of the study conveyed the concern of not being able to use the play center due to sibling intrusion.

Both interventions were feasible to teach parents with the interventionists covering all needed material at the sessions. While the interventions could be taught to parents, and the parents in the HTG clearly understood how to adjust the Just Right Challenges, there were some challenges in adhering to the recommended dose of the intervention. Parents of infants in both the groups demonstrated understanding of the intervention guidelines, however they had difficulty practicing at least 30 minutes of prone play (in play center for infants in the HTG) per day. A parent reported “30 minutes a day is too much and difficult to achieve but . . . we are trying to get through it”. A possible explanation of a high percent of parent-infant dyad not being able to get through at least 30 minutes of prone play could be infants poor tolerance to prone position. The average tolerance of infants to prone position in the beginning of the study was 2.6 minutes for the HTG and 4.2 minutes for the ED. Expecting parents to implement at least 30 minutes of prone play per day in a group of infants with extremely low tolerance for prone might be impractical even when asked to spread the intervention out over multiple times per day.

However, parents in both the groups incorporated prone play into their infant's routine during the study on almost all days they completed the log (93% in HTG and 53% in EG). The total time spent in prone each day increased in both groups. In future studies we will consider making a staged goal for increasing prone. For example 15 minutes in week 1, 20 minutes in week 2, 30 minutes in week 3. Thus parents would be encouraged to reach a target that was consistent with the infants improving prone tolerance. Parent's poor adherence to completing the activity log was a common finding among both the groups. Parents often reported that the activity log is too long and even with the electronic version they tend to miss the notification to complete the activity log on some days during the 3 week intervention period.

To summarize our findings in terms of comparing it to the feasibility threshold set a priori, none of the parents of infants in HTG and EG met the threshold of providing at least 30 minutes of prone play time on 85% of the days during the intervention period. The feasibility threshold of 100% set at parents' ability to identify the “Just Right Challenge” correctly was achieved by parents in the HTG group. While on 50% of the goals were achieved, the intervention remains feasible.

Although the purpose of the study was not to compare the efficacy of the proposed interventions, descriptive group comparisons and effect sizes were calculated to evaluate the sensitivity of the outcome measures to both change over time and detect group differences. Majority of infants in the study had a positive change in their AIMS and GMFM-66 scores. Each of these measures had the same number of infants from the total sample (4 out of 9) that had a change in their scores more than the SDC. This finding suggests that both measures were equally sensitive to change over time for the total sample from baseline to end of intervention. Although a large effect size was found for both the motor development and prone tolerance measures to change over time in the total sample, the lower bound of the CI for GMFM-66 overlapped zero which means that this measures may not be sensitive to change in a 3 week period. In terms of discussing our findings on the sensitivity of the measures to detect group differences, in the HTG, both the AIMS and GMFM-66 exhibited change over time suggesting either measure was sensitive to change in this group. However, only GMFM-66 was sensitive to change over time in the infants in the EG, as no infant in the EG had an increase on the AIMS score more than the SDC. AIMS had a promising effect size and CI compared to GMFM-66. This reflects that the AIMS may be a more sensitive measure to detect group differences in response to the proposed intervention. However, the insensitivity of AIMS in detecting change in motor skill in EG over time as seen by GMFM-66 should not be ignored. Thus it continues to be unclear which measures, the AIMS or GMFM-66, is more sensitive to the changes that can be reported after a 3 week intervention.

The prone tolerance measure developed for this example is a feasible measure to administer; however, the ability of this measure to detect group differences in the change in prone tolerance needs to be investigated more due to the lower bound of CI approaching the value of zero, indicating no real change. An increase in prone tolerance appear to be seen in infants whose parents also reported an increase in prone play duration at home. The 2 infants (infant 2 and infant 3 in Table 5) with the greatest increase in prone tolerance also were reported to spend more than 30 minutes in prone at home more than any other infants and had an increase in the motor skills higher than the SDC on the GMFM-66. These findings, while preliminary, provide support for the use of this measure.

The example's findings show preliminary support for the “active ingredients” of the HTG intervention administered using the instrumented play center (the “active ingredients” being positive reinforcement of infant's efforts to play in prone and encouragement of infant to lift higher in prone). Of the 4 infants who completed the HTG intervention, the 2 infants who used the play center for the longest duration per day and reached the challenging level of the “Just Right Challenges” had the greatest positive, meaningful change in their motor development and prone tolerance (Table 5 and Table 6). As the potential mediators were explored, infant's age, motor skills and prone tolerance were not seen at baseline to modify the relationship between the intervention and infants' motor skills and prone tolerance. However, infants who had a gradual increase in time spent in prone and practiced >30 minutes of prone play at home had the maximum gain in prone tolerance and a real change seen in their motor skills (Table 5). While not conclusive given the feasibility status of this study, the preliminary findings support the theoretical model underpinning the intervention.

The play center device showed promise for improving motor skills in early infancy. During this process it was ensured that parents and infants can utilize the device in their natural environment to maximize the prospects of functional gains in infants.

### Conclusion

This example demonstrates the feasibility of the proposed high technology intervention. An efficacy clinical trial is needed to determine whether this novel intervention has the potential to influence prone tolerance and development in infants with poor prone tolerance and low prone motor skills as well as those at risk for developmental delays.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

While exemplary embodiments of the present invention have been disclosed herein, one skilled in the art will recognize that various changes and modifications may be made without departing from the scope of the invention as defined by the following claims.

We claim:

1. A method for training children with respect to motor control in prone, comprising
monitoring head position of a child in prone; and
activating a positive reinforcement feedback device when the head position is detected as exceeding an adjustable activation threshold.

2. The method of claim 1, wherein the positive reinforcement feedback device is a toy.

3. The method of claim 2, wherein the activating step causes the toy to dance and/or sing.

4. The method of claim 1, further comprising setting the activation threshold by calculating an average height of head lifts using data collected during the monitoring step.

5. The method of claim 1, further comprising
storing data collected during the monitoring step, and
determining changes in child's prone activity over time using the stored data.

6. The method of claim 1, further comprising adjusting the adjustable activation threshold based on a specific child.

7. The method of claim 1, further comprising selectively deactivating the positive reinforcement feedback device after a predetermined time interval if during the time interval the head position has not fallen below the activation threshold and subsequently exceeded the activation threshold.

8. The method of claim 7, wherein the predetermined time interval is adjustable between 0 and 10 seconds.

9. An instrumented play center, comprising
one or more sensors configured or configurable for monitoring head position of a child in prone;
a positive reinforcement feedback device; and
a controller configured to activate the positive reinforcement feedback device when the monitored head position is detected as exceeding an adjustable activation threshold.

10. The instrumented play center of claim 9, wherein the positive reinforcement feedback device is a toy.

11. The instrumented play center of claim 10, wherein the toy is a dancing and/or singing toy when activated.

12. The instrumented play center of claim 9, wherein the one or more sensors include a plurality of ultrasonic sensors.

13. The instrumented play center of claim 9, wherein the controller is configured for adjusting the activation threshold higher over a course of multiple play sessions.

14. The instrumented play center of claim 9, wherein the controller is configured to allow activation threshold adjustments which are child specific.

15. The instrumented play center of claim 9, wherein the controller is configured to operate or be operable in an interval mode by which after activation of the positive reinforcement feedback device, the positive reinforcement feedback device is deactivated after a predetermined time interval if during the time interval the head position has not fallen below the activation threshold and subsequently exceeded the activation threshold.

16. The instrumented play center of claim 15, wherein the predetermined time interval is adjustable between 0 and 10 seconds.

17. A computer program product for training children with respect to motor control in prone, the computer program product comprising a computer readable storage medium having program instructions embodied therewith, the program instructions executable by a device to cause the device to perform a method comprising:
monitoring head position of a child in prone; and
activating a positive reinforcement feedback device when the head position is detected as exceeding an adjustable activation threshold.

18. The computer program product of claim 17, the computer readable storage medium having further program instructions which are executable to cause the device to perform
storing data collected during the monitoring step, and
determining changes in child's prone activity over time using the stored data.

19. The computer program product of claim 17, the computer readable storage medium having further program instructions which are executable to cause the device to perform selectively deactivating the positive reinforcement feedback device after a predetermined time interval if during the time interval the head position has not fallen below the activation threshold and subsequently exceeded the activation threshold.

20. The computer program product of claim 19, wherein the predetermined time interval is adjustable between 0 and 10 seconds.
